(19) United States
(12) Patent Application Publication
(10) Pub. No.: US 2020/0148231 A1
(43) Pub. Date: May 14, 2020

(54) VEHICLE OCCUPANT IMPAIRMENT DETECTION

(71) Applicant: Ford Global Technologies, LLC, Dearborn, MI (US)

(72) Inventors: Ali HASSANI, Ann Arbor, MI (US); Kwaku O. PRAKAH-ASANTE, Commerce Twp, MI (US); David MELCHER, Ypsilanti, MI (US); Jeffrey Allen GREENBERG, Ann Arbor, MI (US); Devinder Singh KOCHHAR, Ann Arbor, MI (US); Jeffrey Brian YEUNG, Canton, MI (US); Kenneth Michael MAYER, Ypsilanti, MI (US)

(21) Appl. No.: 16/621,373

(22) PCT Filed: Jun. 16, 2017
(86) PCT No.: PCT/US2017/037815

§ 371 Date: Dec. 11, 2019

(51) Int. Cl. B60W 60/00; G16H 50/30; G06N 5/04; G05D 1/00; A61B 5/00; A61B 5/0205; A61B 5/18

(52) U.S. Cl. CPC B60W 60/0051; G16H 50/30; G06N 5/047; G05D 1/0061; A61B 5/6833; A61B 5/0205; A61B 5/021; A61B 5/4845; B60W 2540/30; B60W 2540/049; B60W 2540/221; B60W 2540/24; G05D 2201/0213; A61B 5/18

(57) ABSTRACT

A computer is programmed to receive biometric data, from a transdermal patch in a vehicle during operation of a vehicle, wherein the biometric data include a measurement of a chemical. The computer is programmed to actuate a vehicle component, upon determining from a combination of the measurement of the chemical and vehicle operating data that a risk threshold is exceeded.

### BACKGROUND

Impairment, e.g., a lack of alertness, slowed reflexes, dulled senses, etc., of a vehicle user, i.e., occupant, may cause accidents with other vehicles, pedestrians, etc. For example, user impairments can be caused by consumption of chemical substances, e.g., drugs. Consuming chemical substances may cause drowsiness, visual impairment, etc. It is a problem that vehicles lack adequate means to detect vehicle user impairment caused by drug's consumption. Vehicle users or occupants are typically unlikely to report or record their own impairment, but vehicles lack systems to gather, analyze, and act on data that may be indicative of an occupant's impairment.

### BRIEF DESCRIPTION OF THE DRAWINGS

FIG. 1 is a diagram showing a vehicle system for detecting occupant impairment.

FIG. 2 is a block diagram of a transdermal patch.

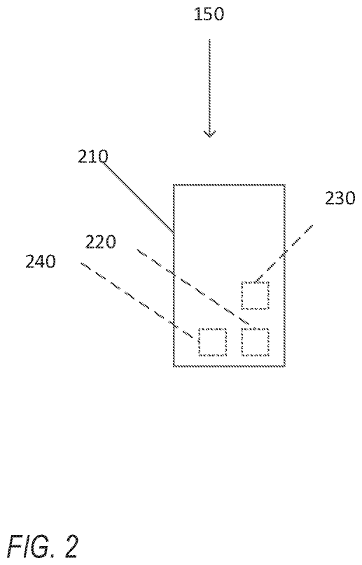

FIG. 3 is a flowchart of an exemplary process for determining an occupant classifier.

FIG. 4 is a flowchart of an exemplary process to detect a vehicle occupant impairment.

### DETAILED DESCRIPTION

### Introduction

Disclosed herein is a computer that is programmed to receive biometric data, from a transdermal patch in a vehicle during operation of a vehicle, wherein the biometric data include a measurement of a chemical. The computer is further programmed to actuate a vehicle component, upon determining from a combination of the measurement of the chemical and vehicle operating data that a risk threshold is exceeded.

The biometric data may further include a heart rate and a blood pressure.

The computer may be further programmed to receive the biometric data from a wearable computing device.

The computer may be further programmed to determine an occupant driving pattern classifier based on the biometric data and the vehicle operating data.

The computer may be further programmed to determine whether the risk threshold is exceeded based on the occupant driving pattern classifier.

The occupant driving pattern classifier may further include a relationship between the biometric data and a driving pattern.

The driving pattern may further include a statistical characteristic related to lane keeping.

The computer may be further programmed to determine a plurality of driving pattern classifiers for a plurality of vehicle occupants, wherein each of the classifiers is associated with one of the plurality of vehicle occupants.

The computer may be further programmed to determine, based on the biometric data, whether there is a lack of an expected chemical, and determine, based on the lack of the expected chemical, whether the risk threshold is exceeded.

Actuating the vehicle component may further include activating an autonomous mode of the vehicle.

The computer may be included in the transdermal patch.

Further disclosed herein is a method that includes receiving biometric data, from a transdermal patch in a vehicle during operation of a vehicle, wherein the biometric data include a measurement of a chemical. The method further includes actuating a vehicle component, upon determining from a combination of the measurement of the chemical and vehicle operating data that a risk threshold is exceeded.

The biometric data may further include a heart rate and a blood pressure.

The method may further include receiving the biometric data from a wearable computing device.

The method may further include determining an occupant driving pattern classifier based on the biometric data and the vehicle operating data.

Determining whether the risk threshold is exceeded may be further based on the occupant driving pattern classifier.

The occupant driving pattern classifier may include a relationship between the biometric data and a driving pattern.

The driving pattern may include a statistical characteristic related to lane keeping.

The method may further include determining, based on the biometric data, whether there is a lack of an expected chemical, and determining, based on the lack of the expected chemical, whether the risk threshold is exceeded.

Actuating the vehicle component may further include activating an autonomous mode of the vehicle.

Further disclosed is a computing device programmed to execute the any of the above method steps. Yet further disclosed is a vehicle comprising the computing device.

Yet further disclosed is a computer program product, comprising a computer readable medium storing instructions executable by a computer processor, to execute any of the above method steps.

### Exemplary System Elements

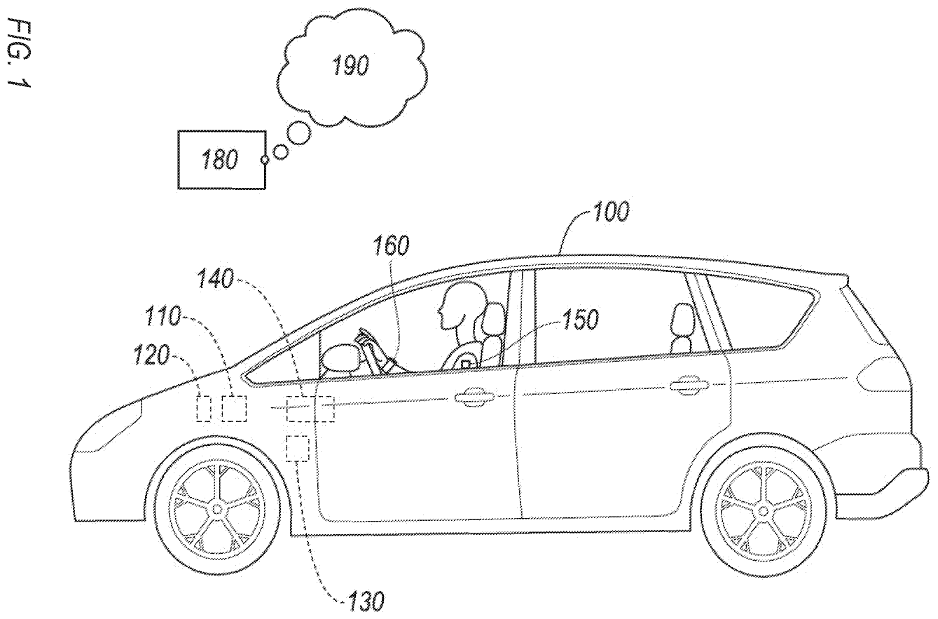

FIG. 1 illustrates a vehicle 100. The vehicle 100 may be powered in a variety of known ways, e.g., with an internal combustion engine, electric motor, etc. Although illustrated as a passenger car, the vehicle 100 may be another kind of powered (e.g., electric and/or internal combustion engine) vehicle such as a truck, a sport utility vehicle, a crossover vehicle, a van, a minivan, etc. The vehicle 100 may include a computer 110, actuator(s) 120, sensor(s) 130, and a human machine interface (HMI 140). In some examples, as discussed below, the vehicle is an autonomous vehicle configured to operate in an autonomous (e.g., driverless) mode, a semi-autonomous mode, and/or a non-autonomous mode.

The computer 110 includes a processor and a memory such as are known. The memory includes one or more forms of computer-readable media, and stores instructions executable by the computer 110 for performing various operations, including as disclosed herein.

The computer 110 may include programming to operate one or more systems of the vehicle 100, e.g., land vehicle brakes, propulsion (e.g., one or more of an internal combustion engine, electric motor, etc.), steering, climate control, interior and/or exterior lights, etc. The computer 110 may operate the vehicle 100 in an autonomous mode, a semi-autonomous mode, or a non-autonomous mode. For purposes of this disclosure, an autonomous mode is defined as one in which each of vehicle propulsion, braking, and steering are controlled by the computer 110; in a semi-autonomous mode the computer controls one or two of vehicle propulsion, braking, and steering; in a non-autonomous mode, a human operator controls the vehicle propulsion, braking, and steering.

The computer 110 may include or be communicatively coupled to, e.g., via a communications bus of the vehicle 100 as described further below, more than one processor, e.g., controllers or the like included in the vehicle 100 for monitoring and/or controlling various controllers of the vehicle 100, e.g., a powertrain controller, a brake controller, a steering controller, etc. The computer 110 is generally arranged for communications on a communication network of the vehicle 100, which can include a bus in the vehicle 100 such as a controller area network (CAN) or the like, and/or other wired and/or wireless mechanisms.

Via the communication network of the vehicle 100, the computer 110 may transmit messages to various devices in the vehicle 100 and/or receive messages from the various devices, e.g., an actuator 120, an HMI 140, etc. Alternatively or additionally, in cases where the computer 110 actually comprises multiple devices, the vehicle communication network may be used for communications between devices represented as the computer 110 in this disclosure.

The actuators 120 of the vehicle 100 are implemented via circuits, chips, or other electronic and/or mechanical components that can actuate various vehicle subsystems in accordance with appropriate control signals, as is known. The actuators 120 may be used to control vehicle systems such as braking, acceleration, and/or steering of the vehicles 100.

In addition, the computer 110 may be configured for communicating through a vehicle-to-infrastructure (V-to-I) interface with other vehicles, and/or a remote computer 180 via a network 190. The network 190 represents one or more mechanisms by which the computer 110 and the remote computer 180 may communicate with each other, and may be one or more of various wired or wireless communication mechanisms, including any desired combination of wired (e.g., cable and fiber) and/or wireless (e.g., cellular, wireless, satellite, microwave and radio frequency) communication mechanisms and any desired network topology (or topologies when multiple communication mechanisms are utilized). Exemplary communication networks include wireless communication networks (e.g., using one or more of cellular, Bluetooth, IEEE 802.11, etc.), dedicated short range communications (DSRC), local area networks (LAN) and/or wide area networks (WAN), including the Internet, providing data communication services.

The HMI 140 may be configured to receive occupant input, e.g., during operation of the vehicle 100. Moreover, an HMI 140 may be configured to present information to a vehicle occupant such as an operator (e.g., driver) and/or passenger. Thus, the HMI 140 is typically located in a passenger cabin of the vehicle 100. For example, the HMI 140 may provide information to the occupant including an indication of vehicle 100 occupant impairment, an activation of vehicle 100 autonomous mode based on vehicle 100 occupant impairment, etc.

The sensors 130 may include a variety of devices known to provide operating data to the computer 110. In the context of this disclosure, vehicle 100 “operating data” means data received from sensors 130 and/or electronic control units (ECUs) in the vehicle describing a state of the vehicle 100 (e.g., speed, a transmission state, etc.) a component thereof, and/or data sensed from a vehicle 100 environment while the vehicle 100 is operating. For example, the sensors 130 may include Light Detection And Ranging (LIDAR) sensor(s) 130 disposed on a top, a pillar, etc. of the vehicle 100 that provide relative locations, sizes, and shapes of other vehicles and/or objects surrounding the vehicle 100. As another example, one or more radar sensors 130 fixed to vehicle 100 bumpers may provide locations of second vehicles travelling in front, side, and/or rear of the vehicle 100, relative to the location of the vehicle 100. The sensors 130 may further alternatively or additionally include camera sensor(s) 130, e.g. front view, side view, etc., providing images from an area around the vehicle 100. For example, the computer 110 may be programmed to receive operating data including image data from the camera sensor(s) 130 and to implement image processing techniques to detect lane markings, traffic signs, and/or other objects such as other vehicles. As another example, the computer 110 may be programmed to determine whether a distance to another vehicle is less than a predetermined threshold, whether an unexpected lane departure occurred, etc. The computer 110 may receive operating data including object data from, e.g., camera sensor 130, and operate the vehicle 100 in an autonomous and/or semi-autonomous mode based at least in part on the received object data. Additionally or alternatively, the operating data may include time-to-collision, average speed, speed variations, occupant reaction time, etc.

The sensors 130 may include a Global Positioning Sensor 130 (GPS). Based on data received from the GPS sensor 130, the computer 110 may determine geographical location coordinates, movement direction, speed, etc., of the vehicle 100. The sensors 130 may include acceleration sensors 130 providing longitudinal and/or lateral acceleration of the vehicle 100.

The computer 110 is programmed to receive occupant biometric data via various devices such as the sensors 130, a transdermal patch 150, a wearable device 160, etc. Biometric data, in the context of present disclosure, is data about a physical state or attribute of an occupant and may include chemical concentrations in occupant bloodstream and/or physiological markers. Chemical concentrations may include chemical levels, e.g., in units of part per million (ppm), of glucose, enzymes, drug substances, etc. in occupant blood. As discussed below, drugs may include prescribed, over-the-counter, and/or illicit drugs such as narcotics. The term “physiological marker” refers to a measurable indicator of some biological state or condition. e.g., a pulse rate, a respiration rate, a body temperature, pupil dilation, etc. Physiological markers may include pupil diameter, heart rate, breadth rate, blood pressure value, reaction time, pupillary response, skin temperature, muscle tremors, etc.

A transdermal patch 150 that is typically used for drug delivery may include sensors to determine various biometric data such as blood content of a chemical substance, etc. A transdermal patch 150 is a medicated adhesive patch that can be placed on the skin to deliver a predetermined dose of medication through occupant's skin and into an occupant bloodstream. Typically, a transdermal patch 150 includes a membrane 210 and a medicine reservoir 220. The patch 150 may further include a sensor 230 and a wireless transceiver 240. The computer 110 may be programmed to receive the biometric data including levels of chemicals in an occupant bloodstream from the patch 150 sensor 230 via the transceiver 240. The computer 110 may be programmed to communicate with the patch 150 via various wireless communication protocols such as Bluetooth™ Low Energy (BLE). In one example, the patch 150 sensor 230 may be capable of determining a concentration of a chemical in the occupant blood with a precision at a microgram order of magnitude.

A wearable device 160 may provide occupant biometric data such as occupant heart rate, body temperature, etc.

As another example, an implantable biomedical device such as a miniaturized robot implanted in occupant's body (e.g. inside blood vessels), a device implanted under the skin, etc. may provide biometric data of the occupant.

The biometric data may include vehicle 100 occupant personal information or profile such as age, height, weight, medical record, etc. The computer 110 may be programmed to receive the occupant profile from, e.g., the remote computer 180 via the communication network 190, a vehicle 100 sensor 130, another computer 110 in the vehicle 100, etc. The medical record may include occupant health condition including any diagnosed physiological and/or mental condition, etc. Additionally or alternatively, the medical record may include information including prescribed and/or over-the-counter drugs. A drug consumption profile may include drug dosage (e.g., 200 milligrams (mg) per capsule), consumption (e.g., 3 capsules/day), etc. Additionally or alternatively, the medical record may include purchase history including over-the-counter drugs, and/or prescribed drugs.

Drugs, in the context of present disclosure, include pharmaceutical drugs, narcotics, etc. Pharmaceutical drugs may include over-the-counter drugs, prescribed drugs, etc. that are typically consumed to cure, treat, and/or prevent a disease, symptom, etc. For example, an epilepsy drug may be consumed by an occupant to prevent a seizure. A blood pressure drug may be consumed to control, e.g., by reducing, an occupant blood pressure within an expected range. Thus, a failure to consume an epilepsy drug, a high blood pressure drug, etc., may cause symptoms such as seizure, high blood pressure, etc. The narcotics may include various types of opioids. A consumption of a narcotic drug may affect mental awareness of a vehicle 100 occupant that may cause cognitive impairment, vision impairment, dizziness, weakness, etc.

With reference to FIG. 1, a computer, e.g., the vehicle 100 computer 110, a computer included in the patch 150, etc., is programmed to receive biometric data from a transdermal patch 150 in a vehicle 100 during operation of the vehicle 100, wherein the biometric data include a measurement of an amount of a chemical in the occupant's body. The computer 110 is further programmed to actuate a vehicle 100 component, upon determining from a combination of the measurement of the chemical and vehicle 100 operating data that a risk threshold is exceeded.

Risk measurements as discussed herein include a value, typically specified by a number, indicating a likelihood of a deviation of and/or an amount of deviation of a vehicle 100 user performance from an expected user performance caused by vehicle 100 user impairment. The expected user performance, in the context of present disclosure, may refer to user performance in controlling vehicle 100 operation including controlling speed, steering, braking, etc. A deviation of expected user performance may be measured according a change in vehicle speed, steering braking, etc., e.g., a lane departure, sudden braking, sudden acceleration, extremely low or high speeds (e.g., more than 25% above or below an established speed limit), etc., may indicate a deviation of expected user performance. As discussed below, the risk may be determined based on a risk classifier. In one example, the risk may be assigned to one of a plurality of discrete categories, such as “low”, “medium”, “high”, and “imminent” risk. A risk level may be correlated to a likelihood of vehicle 100 impact. For example, a “high” level of risk compared to a “low” level of risk may indicate a higher likelihood of vehicle 100 impact. Upon detecting a risk above a threshold, the computer 110 may actuate the vehicle 100 actuators 120 to cause an action such as stopping the vehicle 100, activating a vehicle 100 autonomous mode, etc., if the risk is “high”, i.e., greater than a “medium” risk threshold. In another example, the risk may be defined as a numerical percentage value between 0% and 100%.

The computer 110 may actuate the vehicle 100 actuators 120 to cause an action when the risk, e.g. 60%, is greater than a risk threshold, e.g., 50%. The computer 110 may be programmed to activate a vehicle 100 autonomous mode upon determining that the risk threshold is exceeded. Additionally or alternatively, the computer 110 may be programmed to send a message including, e.g., a vehicle 100 identifier such as a vehicle identification number (VIN) or the like, etc., to the remote computer 180, upon determining that the risk threshold is exceeded. In another example, the computer 110 may be programmed to cause an action assigned to a risk level, e.g., as shown in Table 1.

TABLE 1

| Risk | Action |
|---|---|
| Low | No action |
| medium | Activate semi-autonomous mode, e.g., |
| | activating lane keeping assistance operation |
| High | Activate autonomous mode |
| Imminent | Navigate to side of road and stop the vehicle |

As discussed above, a drug may be consumed by a vehicle 100 occupant to prevent a symptom. For example, an epilepsy drug may be consumed to prevent a seizure. Thus, a lack of consuming an epilepsy drug may indicate a risk of an occupant seizure during driving the vehicle 100. For example, the computer 110 may be programmed to determine, based on the biometric data, whether there is a lack of an expected chemical, and determine, based on the lack of the expected chemical, whether the risk threshold is exceeded.

Consuming more than prescribed dosage of a drug may cause symptoms that impair a vehicle 100 occupant. The computer 110 may be programmed to determine, based on the biometric data, whether there is an overdose of a chemical, and determine, based on the over-dosage of the chemical, whether the risk threshold is exceeded. The computer 110 may be programmed to determine an amount of a deviation of an expected chemical, and determine the risk based on the determined deviation. In one example, the computer 110 may be programmed to determine the risk based on a determined deviation percentage, e.g., as shown in Table 2. A deviation, as the term used herein, includes a difference compared to the expected value, i.e., either under-dosage or over-dosage.

TABLE 2

| Risk | Drug dosage deviation |
|---|---|
| Low | Greater than 5% and less than 10% |
| medium | Greater than 10%, less than 30% |
| High | Greater than 30% |

The remote computer 180 may be programmed to determine an occupant classifier including a chemical pattern classifier and/or a driving pattern classifier. An occupant classifier may be associated with the respective occupant and/or a group of occupants. For example, the remote computer 180 may be programmed to associate a user occupant classifier to an identifier of the respective occupant. Statistical classifiers are generally known. An occupant classifier, as discussed herein, is a set of determined statistical features for an occupant, e.g., average values that then are used to classify the occupant according to one or more categories, e.g., impaired or not impaired, high, medium or low risk level due to drug consumption, etc. The chemical pattern classifier may include average values, maximum allowed values, etc. for chemicals in occupant's blood. The driving pattern classifiers, as discussed below, refer to statistical features associated with an occupant driving pattern included in vehicle 100 operating data. Table 3 shows an example occupant classifier for one example occupant. In other words, Table 3 shows values identified for the example occupant based on received data associated with the example occupant. The remote computer 180 may be programmed to determine the occupant classifier based on data received from one or more vehicles 100. Additionally, the remote computer 180 may be programmed to receive the biometric data such as occupant age, gender, prescribed drugs, expected dosage, etc. from other computers. In one example, the remote computer 180 may be programmed to store occupant classifiers of multiple occupants in a computer 180 memory. Each of the stored classifiers may be associated with an occupant identifier.

TABLE 3

| | Data associated with the |
|---|---|
| Occupant classifier | example occupant |
| Chemical pattern classifier | |
| Epilepsy drug | Expected dosage between |
| | 2 and 3 ppm |
| Vitamin D | Expected dosage between |
| | 1 and 2 ppm |
| Opioids | Maximum expected value |
| | 1 ppm |
| Physiological markers | |
| Heart rate | Between 70 and 80 beats |
| | per minutes |
| Driving pattern classifier | |
| Number of unexpected | Maximum 2 in 100 km |
| lane departure | |
| Reaction time | Maximum 0.5 |
| Speed | Average between 10% |
| | below and above speed |
| | limit |

Consumption of a drug may not have an effect on a vehicle 100 occupant driving capability. For example, a lack of and/or over-dosage of a supplement such as Vitamin D may not cause a vehicle 100 occupant impairment. The computer 110 may be programmed to receive medical record of a vehicle 100 occupant from a remote computer and to score the drugs based on an effect caused by the drug on occupant driving capability. The score as that term is used herein is a value, e.g., specified by a number between 0 and 10, indicating a relevance of drug to driving impairment. For example, a score of 1 may indicate a lower relevance of a drug, e.g., Vitamin D supplement. In another example, a score of 9 may indicate a higher relevance of a drug, e.g., an epilepsy drug, an opioid, etc.

The computer 110 may be programmed to select a drug upon determining that the score of the drug exceeds a predetermined risk threshold value, e.g., 5, and determine the risk of a selected drug based on the deviation of drug expected dosage, e.g., Table 2. For example, a narcotic concentration, e.g., opioids, in a vehicle 100 occupant's blood may be expected to be below 1 ppm. The narcotics may cause cognitive impairment, i.e., having a high risk, e.g., 8, as discussed above. Thus, a concentration of 1.5 ppm may be 50% more than a maximum expected concentration. Thus, the computer 110 may be programmed to determine a high risk upon determining that an occupant blood has a 1.5 ppm concentration of narcotics.

As discussed above, the biometric data may include the physiological markers such as a heart rate, a blood pressure, etc. of a vehicle 100 occupant. An unexpected physiological marker indicator, e.g., high heart rate, may indicate an occupant impairment. In other words, the risk may be determined based on a deviation of a physiological marker from an expected value and/or an expected range. However, expected ranges of physiological markers are typically wide enough to make a deviation detection for a specific occupant difficult. For example, expected range of heart rate for an adult human is between 60 to 100 beats per minute. In order to be able to precisely detect a deviation of a physiological marker, an expected value for each vehicle 100 occupant may be used. In one example, the computer 110 may be programmed to receive data including average expected value of physiological markers, e.g., a heart rate of 75 beats/second, for each of vehicle 100 occupants. The computer 110 may be programmed to determine a deviation of a physiological marker for an occupant based on received average expected value of the physiological marker for the respective occupant. The computer 110 may be programmed to determine the risk associated with a vehicle 100 occupant based on the determined deviation of the physiological marker from an average expected value for the respective occupant, e.g., based on Table 2.

As discussed above, the risk may be determined based on a deviation of an occupant physiological marker from an expected value and/or a deviation of expected concentration of a chemical in occupant's blood. However, a deviation of chemical and/or a deviation of a physiological marker may cause different effects in different occupants. For example, a 30% deviation of a heart rate from an expected value may cause different changes in two different occupants. It may cause 50% increase in reaction time of a first occupant and only 20% in a reaction time of a second occupant. Thus, the computer 110 may be programmed to determine whether the risk threshold is exceeded further based on a driving pattern classifier, e.g., Table 2.

The computer 110 may be programmed to determine multiple driving pattern classifiers for respective vehicle 100 occupants. Each of the classifiers may be associated with one of the vehicle 100 occupants. The computer 110 may be programmed to create an occupant driving pattern classifier based on the biometric data and the vehicle 100 operating data. In one example, the computer 110 may be programmed to determine an average expected value for each of multiple vehicle 100 operating data, e.g., an average speed, average reaction time, etc.

In one example, a driving pattern of a vehicle 100 occupant includes a statistical characteristic related to lane keeping, e.g., a maximum expected number of unexpected lane departure such as 1 unexpected departure per hour, 2 unexpected departure per 100 kilometers, etc. The computer 110 may be programmed to determine the average vehicle 100 operating data based on received sensor 130 data over a predetermined period and/or driven distance, e.g., 1 month, 1000 kilometers (km), etc. The computer 110 may be programmed to determine an occupant driving pattern based on the received vehicle 100 operating data.

As discussed above, in one example, the computer 110 can be programmed to determine the risk based on received biometric data. In another example, the risk may be determined based on vehicle 100 operating data. Thus, in yet another example, the computer 110 may be programmed to determine classifiers that include a relationship between the biometric data and a driving pattern. In other words, the computer 110 may be programmed to determine the risk based on a combination of a determined deviation or differences of biometric data and the operating data, e.g., aggregations or sums of differences, deviations of statistical measures derived biometric and operating data, etc.

For example, the computer 110 may be programmed to determine the risk based on a sum of the deviations, e.g., a “high” level of risk when a sum of deviations exceeds a threshold of 50%. For example, the computer 110 may determine a risk to be at a “high” level when the computer 110 determines a biometric data (e.g., heart rate) deviation of 20% and an operating data (e.g., a number of unexpected lane changes) deviation of 35%, because the sum of deviations, i.e., 55%, is greater than the threshold of 50%.

In another example, the computer 110 may be programmed to determine the risk based on a risk classifier. The risk classifier may include a mathematical operation such as a₁X₁+a₂X₂+b₁Y₁+b₂Y₂. The result of this operation can provide a risk value that can then be used to classify a risk associated with an occupant based on current data. In the foregoing example expression, X₁, X₂, etc., represent biometric data, e.g., a deviation of expected chemical concentration on occupant's blood. For example, X₁ may be 50% when a drug concentration of 1.5 ppm is measured while a concentration of 1 ppm is expected based on the user classifier. Further, Y₁, Y₂, etc., represent vehicle 100 sensor 130 data such as a deviation from average expected speed, acceleration, etc. The parameters a₁, a₂, etc., and b₁, b₂, etc. may be optimized to define the risk classifier. In one example, the computer 110 may be programmed to determine optimized parameters a₁, a₂, etc., an b₁, b₂, etc. using artificial intelligence and/or other known optimization techniques such as genetic algorithms

The computer 110 may be programmed to perform an action such as actuating a vehicle 100 component upon determining that the risk calculated based on the risk classifier exceeds a risk threshold. For example, the computer 110 may be programmed to cause an action assigned to a risk level, e.g., as shown in Table 1. The computer 110 may activate a vehicle 100 semi-autonomous mode, e.g., controlling a vehicle 100 steering operation, upon determining a medium risk. Upon determining a high risk, the computer 110 may activate a vehicle 100 autonomous mode to navigate the vehicle 100 to a vehicle 100 destination. Upon determining an imminent risk, the computer 110 may activate a vehicle 100 autonomous mode to navigate the vehicle 100 to a road side, e.g., nearest possible road side where the vehicle 100 can stop, and stop the vehicle 100.

### Processing

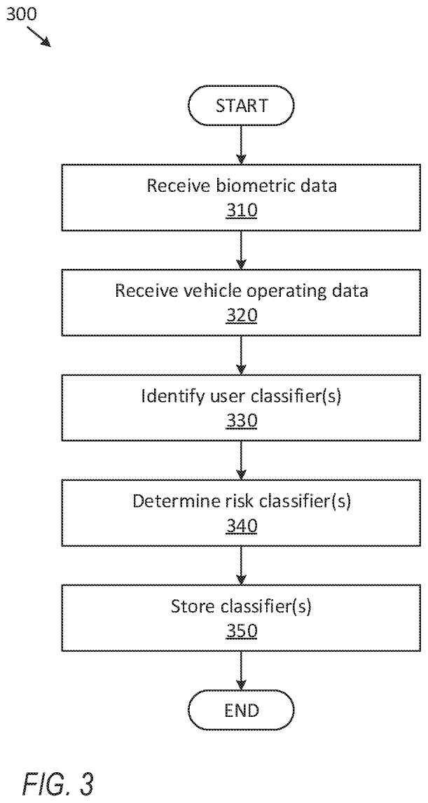

FIG. 3 is a flowchart of an exemplary process 300 for determining an occupant classifier. For example, the remote computer 180, the vehicle 100 computer 110, a combination thereof, etc., can be programmed to execute blocks of the process 300.

The process 300 begins in a block 310, in which the remote computer 180 receives biometric data of one or more vehicle 100 occupants. The remote computer 180 may be programmed to receive the data via the wireless communication network 190 from one or more vehicles 100. The biometric data may include occupant medical record, prescribed drugs, etc. Additionally, the biometric data may include a concentration of one or more chemicals in occupant's blood, one or more physiological markers such as hear rate, blood pressure, etc.

Next, in a block 320, the remote computer 180 receives vehicle operating data. The remote computer 180 may be programmed to receive the vehicle 100 operating data via, e.g., the wireless communication network 190, from one or more vehicles 100.

Next, in a block 330, the remote computer 180 identifies occupant classifier(s). For example, the remote computer 180 may be programmed to identify occupant classifiers for multiple occupants based on data received from one or more vehicles 100. The remote computer 180 may associate an occupant profile to a respective occupant.

Next, in a block 340, the remote computer 180 determines a risk classifier, e.g., as described above. For example, the remote computer 180 may determine a risk classifier based on deviations of the received biometric data and the vehicle 100 operating data from expected values included in occupant's classifier(s).

Next, in a block 350, the remote computer 180 stores the occupant classifiers and/or the risk classifier, e.g., in a remote computer 180 memory. Additionally or alternatively, the remote computer 180 may be programmed to transmit data including the classifiers via the wireless communication network 190 to the vehicle(s) 100. Following the block 350, the process 300 ends, or alternatively returns to the block 310, although not shown in FIG. 3.

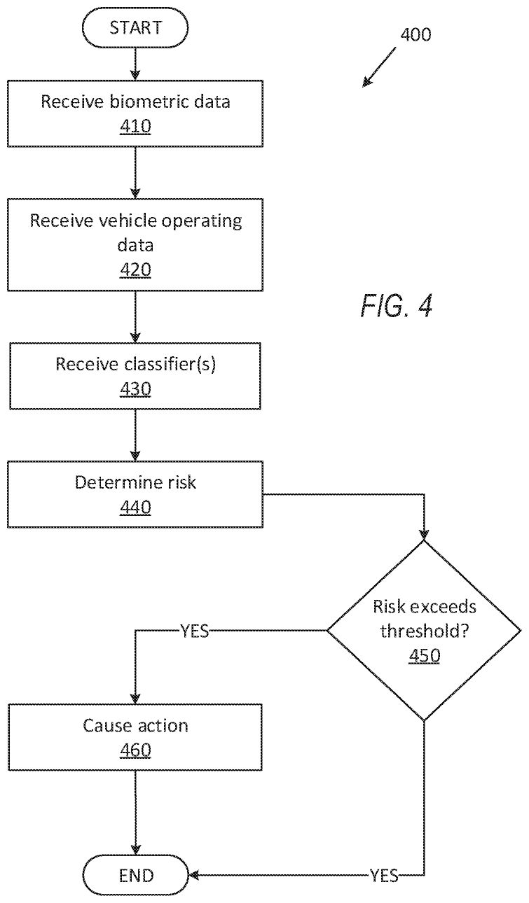

FIG. 4 is a flowchart of an exemplary process 400 to detect a vehicle 100 occupant impairment caused by drug(s). For example, the vehicle 100 computer 110 may be programmed to execute blocks of the process 400.

The process 400 begins in a block 410, in which the computer 110 receives vehicle 100 occupant biometric data. The computer 110 may be programmed to receive the biometric data, e.g., a concentration indicator of a chemical in occupant's blood, of a vehicle 100 occupant from various devices such as a transdermal patch 150, a wearable device 160, a vehicle 100 sensors 130, etc.

Next, in a block 420, the computer 110 receives vehicle 100 operating data. For example, the computer 110 may be programmed to receive a number of unexpected lane departure, a current reaction time of the occupant, speed variations, etc.

Next, in a block 430, the computer 110 receives classifiers. In one example, the computer 110 receives multiple occupant classifiers and/or a risk classifier from the remote computer 180.

Next, in a block 440, the computer 110 determines a risk based on the received biometric data, the received vehicle 100 operating data, and the stored classifiers. For example, the computer 110 may be programmed to determine a deviation of biometric data based on the received biometric data and the occupant classifier, and to determine a deviation of operating data based on the received vehicle 100 operating data and the occupant classifier. The computer 110 may be further programmed to determine the risk based on the determined deviations and the received risk classifier. In one example, the risk classifier may include a sum operation of the determined deviations in percentage, as discussed above.

Next, in a decision block 450, the computer 110 determines whether the determined risk exceeds a predetermined threshold, e.g., 50%. If the computer 110 determines that the risk exceeds the threshold, the process 400 proceeds to a block 460; otherwise the process 400 ends, or alternatively returns to the block 410.

In the block 460, the computer 110 causes an action based on the determined risk. For example, the computer 110 may activate vehicle 100 actuators 120 based on an action assigned to a risk level, e.g., as shown in Table 1 above. Following the block 460, the process 400 ends, or alternatively returns to the block 410, although not shown in FIG. 4.

The article “a” modifying a noun should be understood as meaning one or more unless stated otherwise, or context requires otherwise. The phrase “based on” encompasses being partly or entirely based on.

Computing devices as discussed herein generally each include instructions executable by one or more computing devices such as those identified above, and for carrying out blocks or steps of processes described above. Computer-executable instructions may be compiled or interpreted from computer programs created using a variety of programming languages and/or technologies, including, without limitation, and either alone or in combination, Java™, C, C++, Visual Basic, Java Script, Perl, HTML, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media. A file in the computing device is generally a collection of data stored on a computer readable medium, such as a storage medium, a random access memory, etc.

A computer-readable medium includes any medium that participates in providing data (e.g., instructions), which may be read by a computer. Such a medium may take many forms, including, but not limited to, non-volatile media, volatile media, etc. Non-volatile media include, for example, optical or magnetic disks and other persistent memory. Volatile media include dynamic random access memory (DRAM), which typically constitutes a main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH, an EEPROM, any other memory chip or cartridge, or any other medium from which a computer can read.

With regard to the media, processes, systems, methods, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of systems and/or processes herein are provided for the purpose of illustrating certain embodiments, and should in no way be construed so as to limit the disclosed subject matter.

Accordingly, it is to be understood that the present disclosure, including the above description and the accompanying figures and below claims, is intended to be illustrative and not restrictive. Many embodiments and applications other than the examples provided would be apparent to those of skill in the art upon reading the above description. The scope of the invention should be determined, not with reference to the above description, but should instead be determined with reference to claims appended hereto and/or included in a non-provisional patent application based hereon, along with the full scope of equivalents to which such claims are entitled. It is anticipated and intended that future developments will occur in the arts discussed herein, and that the disclosed systems and methods will be incorporated into such future embodiments. In sum, it should be understood that the disclosed subject matter is capable of modification and variation.

What is claimed is:

1. A computer, programmed to:
receive biometric data, from a transdermal patch in a vehicle during operation of a vehicle, wherein the biometric data include a measurement of a chemical; and
upon determining from a combination of the measurement of the chemical and vehicle operating data that a risk threshold is exceeded, actuate a vehicle component.

2. The computer of claim 1, wherein the biometric data further include a heart rate and a blood pressure.

3. The computer of claim 1, further programmed to receive the biometric data from a wearable computing device.

4. The computer of claim 1, further programmed to determine an occupant driving pattern classifier based on the biometric data and the vehicle operating data.

5. The computer of claim 4, further programmed to determine whether the risk threshold is exceeded based on the occupant driving pattern classifier.

6. The computer of claim 4, wherein the occupant driving pattern classifier further includes a relationship between the biometric data and a driving pattern.

7. The computer of claim 6, wherein the driving pattern includes a statistical characteristic related to lane keeping.

8. The computer of claim 1, further programmed to determine a plurality of driving pattern classifiers for a plurality of vehicle occupants, wherein each of the classifiers is associated with one of the plurality of vehicle occupants.

9. The computer of claim 1, further programmed to determine, based on the biometric data, whether there is a lack of an expected chemical, and determine, based on the lack of the expected chemical, whether the risk threshold is exceeded.

10. The computer of claim 1, wherein actuating the vehicle component further includes activating an autonomous mode of the vehicle.

11. The computer of claim 1, wherein the computer is included in the transdermal patch.

12. A method, comprising:
receiving biometric data, from a transdermal patch in a vehicle during operation of a vehicle, wherein the biometric data include a measurement of a chemical; and
upon determining from a combination of the measurement of the chemical and vehicle operating data that a risk threshold is exceeded, actuating a vehicle component.

13. The method of claim 12, wherein the biometric data further include a heart rate and a blood pressure.

14. The method of claim 12, further comprising receiving the biometric data from a wearable computing device.

15. The method of claim 12, further comprising determining an occupant driving pattern classifier based on the biometric data and the vehicle operating data.

16. The method of claim 15, wherein determining whether the risk threshold is exceeded is further based on the occupant driving pattern classifier.

17. The method of claim 15, wherein the occupant driving pattern classifier includes a relationship between the biometric data and a driving pattern.

18. The method of claim 17, wherein the driving pattern includes a statistical characteristic related to lane keeping.

19. The method of claim 12, further comprising determining, based on the biometric data, whether there is a lack of an expected chemical, and determining, based on the lack of the expected chemical, whether the risk threshold is exceeded.

20. The method of claim 12, wherein actuating the vehicle component further includes activating an autonomous mode of the vehicle.
